(19) United States
(12) Patent Application Publication
(10) Pub. No.: US 2026/0355015 A1
(43) Pub. Date: Nov. 12, 2026

(54) SYSTEMS AND METHODS FOR TREATING EXCESS PRESSURE HAVING AN ELASTIC MEMBRANE IN FLUID CONTACT WITH BODILY FLUID

(71) Applicant: ECOLE POLYTECHNIQUE FEDERALE DE LAUSANNE (EPFL), Lausanne (CH)

(72) Inventor: Nikolaos STERGIOPULOS, Preverenges (CH)

(73) Assignee: ECOLE POLYTECHNIQUE FEDERALE DE LAUSANNE (EPFL), Lausanne (CH)

(21) Appl. No.: 19/720,453

(22) Filed: Jun. 25, 2026

Related U.S. Application Data
(63) Continuation of application No. PCT/IB2024/063324, filed on Dec. 31, 2024.
(60) Provisional application No. 63/616,943, filed on Jan. 2, 2024.

(51) Int. Cl. A61F 9/007; A61M 27/00

(52) U.S. Cl. CPC A61F 9/00781; A61M 27/006

(57) ABSTRACT

Systems and methods are provided for treating diseases that produce elevated pressure, e.g., intraocular pressure or cerebrospinal fluid pressure, such as glaucoma or hydrocephalus, wherein the device includes a housing shell, an elastic membrane, and a local constriction that reduces the area of the fluidic passageway through the housing shell and increases fluidic resistance of the flow of fluid through the passageway. At normal IOP levels, the elastic membrane engages the local constriction to prevent or reduce fluid flow across the passageway. When the average pressure between the inlet and outlet of the device varies, e.g., exceeds a predetermined threshold, the elastic membrane deforms and disengages the local constriction to thereby permit fluid flow across the passageway.

### CROSS-REFERENCE TO RELATED APPLICATIONS

This application is a continuation of International PCT Patent Application No. PCT/IB2024/063324, filed Dec. 31, 2024, which claims priority to U.S. Provisional Patent Application No. 63/616,943, filed Jan. 2, 2024, the entire contents of each of which are incorporated herein by reference.

### FIELD OF USE

The present technology is directed to systems and methods for draining excess bodily fluid, e.g., intraocular fluid and/or cerebrospinal fluid, to maintain pressure within physiological limits, for example, for treating glaucoma and/or hydrocephalus.

### BACKGROUND

Glaucoma affects about 70 million people worldwide, and is a disorder associated with high pressure in the eye resulting from the generation of excess intraocular fluid (aqueous humor). Aqueous humor is produced at a rate of 2-3 μl/min by the ciliary body and in a normal human eye maintains a constant intraocular pressure (“IOP”) around 12-20 mmHg. Aqueous humor exits the eye primarily through the trabecular meshwork and Schlemm's canal, where it eventually drains to the episcleral veins. Maintaining intraocular pressure within appropriate ranges is critical to health of the eye, and depends on aqueous humor dynamics, namely the production rate from the ciliary body (aqueous humor inflow) and its outflow rate through the trabeculum. The most frequent type of glaucoma, called open-angle glaucoma, results from an increase in the fluidic resistance of the trabecular meshwork. Left untreated, this disease typically causes damage to the optic nerve, with consequent loss of vision, initially peripheral, but progressively leading to total blindness. Unfortunately, glaucoma is often asymptomatic until late in the progress of the disease.

Traditionally, glaucoma is treated using medication, for example, the daily application of eye drops, such as Brinzolamide ophthalmic, that reduce production of aqueous humor. Such medications do not cure glaucoma, and must be continued to be taken to maintain intraocular pressures within accepted limits. In certain cases, such treatment may fail, and other surgical treatments are employed, such as filtering procedures or placement of a glaucoma drainage device. Glaucoma drainage devices reduce intraocular fluid pressure by providing an artificial drainage pathway, thus maintaining a low IOP.

Previously-known glaucoma drainage devices usually comprise a structure having a drainage tube that is inserted through a small incision made in the conjunctiva. The surgeon makes a tiny incision in the sclera of the eye and creates an opening for the drainage implant device. The drainage tube is placed such that the opening of the tube is disposed in the anterior chamber of the eye within the aqueous humor. The tube is sutured in place with the drainage device attached to the sclera of the eye. Many surgeons will place an absorbable suture around the tube at the time of surgery to prevent over-filtration through the device until a fibrous capsule has formed. Accordingly, such devices typically are not functional until about 3 to 8 weeks after the procedure, so as to prevent over-filtration.

An exemplary previously-known passive glaucoma drainage device is described in U.S. Pat. No. 4,457,757 to Molteno. The device described in that patent comprises a tube of a biologically inert silicone configured to be inserted into the eye to drain aqueous humor from the anterior chamber of the eye. The device does not include a pressure regulating mechanism, but instead relies on the resistance to aqueous flow through the tubing to prevent over drainage.

One drawback of devices such as those described in the Molteno patent is that the drainage flow depends on IOP and on the fixed hydrodynamic resistance of the shunt. In many cases, however, the hydrodynamic resistance of the shunt may not be sufficient to reduce high IOP when the resistance to flow is too high, or may lead to over-drainage if the resistance is low. For example, a common problem, which arises shortly after implantation, is hypotony, which occurs when IOP drops below acceptable physiological levels (i.e., IOP <6 mmHg). Hypotony usually takes place the first few days to weeks following the implantation of a glaucoma drainage device, and is a combined result of a low fluidic resistance of both the implant and the distal outflow paths. Hypotony may lead to a number of undesirable effects and complications, such as hypotensive maculopathy, choroidal detachment, etc. Another problem, also related to the fixed fluid resistance of previously known implants, is fibrosis, which appears progressively at long term and which, depending on its extent and severity, may raise the effective fluidic resistance of the implant, thereby raising the IOP to different, often non-physiological, levels.

The foregoing drawbacks have been recognized in the prior art, and several improvements have been attempted to improve flow control over the entirely passive system described in Molteno. For example, U.S. Pat. No. 5,411,473 to Ahmed describes a drainage device that includes a membrane-type valve. More specifically, Ahmed describes a drainage system including a membrane folded and held in tension between two plates to provide a slit opening, such that the membrane responds to pressure changes to open or close the slit opening. Unfortunately, the operational characteristics of the system depend on the properties of the membrane, which cannot be changed easily once the device is implanted. Also, the valve of Ahmed does not provide a true opening pressure to accurately control post-operation IOP.

U.S. Pat. No. 6,544,208 to Ethier describes a self-regulating pressure system. More specifically, Ethier describes an implantable shunt device having a flexible tube positioned in a pressurized enclosure. In this patent, flow through the tube is dependent on a differential pressure between a pressure in the flexible tube and a pressure outside the flexible tube in the pressurized enclosure. However, one skilled and experienced in the field of medical implants, especially in ophthalmology, would understand that such a system with a constant external pressure chamber would be very impractical, if not impossible, to make.

Ethier further describes that the pressure outside the flexible tube in the pressurized enclosure of the implantable shunt device is generated by osmotic effects. More specifically, the pressurized enclosure is filled with a solution containing a solute that generates an osmotic pressure which controls the opening pressure of the implantable shunt device. The implantable shunt device includes a semi-permeable membrane affixed between support gratings that reduce deformation of the semi-permeable membrane. Unfortunately, significant deformation of the semi-permeable membrane makes it difficult to predict the osmotic pressure within the pressurized enclosure. Moreover, such a device with a pressurized enclosure may not work in higher altitudes where there is a relatively large change in atmospheric pressure.

U.S. Pat. No. 9,101,445 to Bigler describes an ocular drainage system for treating diseases that produce elevated intraocular pressures, such as glaucoma, wherein the system includes an implantable device and an external control unit. The implantable device includes a non-invasively adjustable valve featuring at least one deformable tube and a disk rotatably mounted within a housing, such that rotation of the disk using the external control unit causes the disk to apply a selected amount of compression to the deformable tube, thereby adjusting the fluidic resistance of the deformable tube and regulating the intraocular pressure.

Still other examples of previously-known systems are known. U.S. Pat. Nos. 5,626,558 and 6,508,779 to Suson describe a shunt which may be adjusted after implantation by using a low power laser to drill additional openings in the tube wall to adjust the flow rate. U.S. Pat. No. 6,186,974 to Allan et al. describes a drainage shunt having multiple layers, one of which may be a gel that swells upon absorption of fluid to adjust flow rate through the tube. U.S. Pat. No. 6,726,664 to Yaron describes a drainage tube including a distal hook that retains the distal end of the implant within the anterior chamber of the eye, and various means, such as rods or sutures, for partially occluding the lumen of the tube to regulate flow.

Other previously-known glaucoma treatment systems include significantly greater complexity to address the drawbacks of the simpler shunt systems described above. For example, U.S. Pat. No. 6,077,299 to Adelberg, et al. describes a non-invasively adjustable valve implant for the drainage of aqueous humor for treatment of glaucoma, wherein an implant having an inlet tube is surgically inserted in the anterior chamber of the eye to allow aqueous humor to flow from the anterior chamber to a valve. After passing through a pressure and/or flow regulating valve in the implant, the fluid is dispersed along the periphery of the implant to the interior of the Tenon's capsule where it is absorbed by the body. In one embodiment, the valve inhibits flow below, and allows flow above, a specific pressure difference between the IOP within the eye and the pressure within the bleb cavity in the Tenon's capsule. The specified pressure difference or set-point is always positive and the valve is always closed in the presence of negative pressure differences, to prevent reverse flow of fluid from the Tenon's capsule back into the anterior chamber of the eye.

In Adelberg, the valve is formed by a chamber to which the inlet tube is connected, such that the chamber is closed by a pressure sensitive valve in the shape of a flat cone. The pressure regulation set point of the valve is governed by a flexible diaphragm that cooperates with an armature plate having an inclined surface, and which is configured to slide over a complementary inclined surface attached to the diaphragm. Cooperation of the inclined surface of the plate and the complementary surface causes the diaphragm to deflect depending on where the armature plate is located. The armature plate is rotated, using a rotor and a set of speed-reducing and torque-enhancing gears, to regulate the flow through the device. The characteristics of the valve strongly depend on the configuration of the cone shaped valve. In addition, the regulating mechanism is complex, including many rotating parts and gears, and this complexity poses a risk of malfunction.

U.S. Patent Application Pub. No. 2013/0211312 to Gelvin discloses a drainage device for implantation in the eye. A flexible membrane is disposed within the housing of the drainage device. Such a device, however, would not be suitable for a person that travels to different altitudes as the pressure surrounding the device would change.

In view of the drawbacks of the foregoing prior art devices and methods, it would be desirable to provide an ocular drainage system and methods that are capable of maintaining a constant, or nearly constant, IOP, regardless of atmospheric pressure changes, e.g., due the patient's altitude.

It further would be desirable to provide an ocular drainage system having a small volume to facilitate implantation of the device beneath the conjunctiva, either under a relatively small scleral flap or on the scleral surface, or even within a diffuser plate, and having few moving parts, thereby enhancing robustness of the system and reducing the risk of failure arising from operation of complex mechanisms.

Improved ocular drainage systems are described in U.S. Pat. Nos. 10,596,035, 11,779,489, and 12,005,000 to Stergiopulos, the entire contents of each of which are incorporated herein by reference.

In addition, it would be desirable to provide an ocular drainage system that is suitable to a patient that travels to different altitudes.

It further would be desirable to provide an ocular drainage system effective to prevent hypotony post-implantation and/or effective in light of the development of fibrosis at long term.

Moreover, hydrocephalus, also known as “water on the brain,” is a condition where cerebrospinal fluid (CSF) builds up in the brain's ventricles, causing pressure to increase in the skull. CSF is normally produced and absorbed by the body, which protects and nourishes the brain and spinal cord; however, when the flow of CSF is disrupted, the fluid can build up and cause the ventricles to widen, thereby preventing the brain from functioning properly. Current efforts to treat hydrocephalus involve the use of hydrocephalus valves configured to shunt excess CSF from the brain ventricles to other parts of the body, such as the peritoneal cavity, in order to keep pressure in the ventricles of the brain within physiological limits and restore the enlarged ventricles to their normal size. For example, the two main types of hydrocephalus valves are fixed-pressure valves and adjustable-pressure valves.

Fixed-pressure valves have a predetermined opening pressure, such that they allow CSF to drain only when the pressure in the brain exceeds a fixed threshold. While fixed-pressure valves are simple and cost-effective, do not require external adjustments, and are reliable for patients with stable CSF dynamics, such valves lack adaptability as pressure settings cannot be changed post-surgery, and further may require replacement if the patient's CSF dynamics change over time. Commercially available fixed-pressure valves include the Delta Valve (made available by Medtronic, Minneapolis, Minnesota) and Integra VP fixed-pressure valves (made available by Integra LifeSciences, San Diego, California).

On the other hand, adjustable-pressure valves are configured to allow for non-invasive adjustment of the opening pressure, e.g., using an external magnetic or mechanical tool, thereby enabling improved customization to the patient's needs. While adjustable-pressure valves have the flexibility to adapt to changes in CSF dynamics over time, and reduces the need for additional surgeries to modify the pressure settings, such valves are typically more expensive, and the use of magnetic adjustments may interfere with MRI procedures. Commercially available fixed-pressure valves include the Codman Hakim Programmable Valve (made available by Integra LifeSciences, San Diego, California), the proGAV valve (made available by Christoph Miethke GmbH & Co. KG, Potsdam, Germany), and the Polaris and Sophy Mini valves (made available by Sophysa USA, Crown Point, Indiana).

The main drawbacks of programmable valves include their relatively large sizes (e.g., the Codman Hakim Programmable Valve is 18 mm in diameter and 5 mm in thickness), magnetic mechanisms for providing adjustability which may interfere with MRI imaging, and the requirement of a separate anti-siphoning mechanism. For example, because patients change their posture all the time during daily life (e.g., standing up from supine position), the changes in hydrostatic pressure between the proximal site (e.g., brain ventricles) and the distal exit point (e.g., peritoneum) can be very big (e.g., 50 cm H2O, or more), such that the hydrocephalus shunts typically require a separate anti-siphoning mechanism.

It would be desirable to provide an improved CSF drainage system that does not require magnetic adjustments and/or a separate anti-siphoning mechanism.

### SUMMARY

The present technology overcomes the drawbacks of previously-known excessive fluid pressure treatment systems, e.g., ocular drainage systems, by providing an implantable device for the treatment of excess fluid pressure within a bodily area as described herein. In accordance with one aspect, a device for the treatment of excess fluid pressure within a patient is provided. The device may comprise an inlet configured to be implanted to be in fluid communication with a first bodily area, an outlet configured to be implanted in fluid communication with a second bodily area, and an elastic membrane disposed between the inlet and the outlet and configured to be implanted at an implantation site within the patient. The elastic membrane may comprise an interior surface that defines a fluidic channel that permits a flow of fluid from the inlet to the outlet, and an outer surface configured to be in direct contact with fluid or tissue surrounding the device at the implantation site. The elastic membrane may be configured to deform to change a volume of the fluidic channel when an average pressure between the inlet and the outlet varies, thereby varying fluidic resistance of the flow of fluid through the fluidic channel.

In addition, the device may comprise a housing defining the inlet and the outlet. The housing may be coupled to the elastic membrane to define the fluidic channel within the housing In some embodiments, the elastic membrane may be disposed within a cavity of the housing, the cavity comprising one or more openings configured to expose the outer surface of the elastic membrane within the cavity to fluid or tissue surrounding the device at the implantation site. In some embodiments, the first bodily area may comprise an anterior chamber of an eye of the patient, and the second bodily area may comprise a space in a distal portion or an orbital fat space of the eye, such that the elastic membrane may be configured to deform to change the volume of the fluidic channel when the average pressure between the inlet and the outlet varies, thereby varying fluidic resistance of the flow of intraocular fluid through the fluidic channel, e.g., to thereby treat glaucoma. Accordingly, the housing may have a radius of curvature selected to accommodate a radius of curvature of the eye. Moreover, the housing may be configured to be implanted under a conjunctiva. In addition, the device may include a nozzle having an outlet end coupled to the inlet, and an inlet end configured to pass through a wall of an eye to communicate with the anterior chamber of the eye. The device further may include a drainage tube having a proximal end coupled to the outlet, and a distal region configured to be disposed within the space in the distal portion or the orbital fat space of the eye. The distal region of the drainage tube may comprise one or more drainage holes. In some embodiments, the device further may include a diffuser plate having a groove configured to receive a portion of the distal region of the drainage tube. For example, the diffuser plate may be configured to be disposed on a scleral surface of the eye.

In accordance with another aspect, the first bodily area may comprise a ventricle of a brain of the patient, such that the elastic membrane may be configured to deform to change the volume of the fluidic channel when the average pressure between the inlet and the outlet varies, thereby varying fluidic resistance of the flow of cerebrospinal fluid through the fluidic channel, e.g., to thereby treat hydrocephalus. The device further may include a nozzle having an outlet end coupled to the inlet, and an inlet end configured to be disposed within the ventricle of the brain. Moreover, the device may include a drainage tube having a proximal end coupled to the outlet, a distal region configured to pass through a wall of a peritoneum to communicate with a peritoneal cavity of the patient, and a length selected such that the distal region of the drainage tube extends from the outlet to the peritoneal cavity. The distal region of the drainage tube may comprise one or more drainage holes.

The device further may include a local constriction disposed within at least a portion of the fluidic channel to reduce the area of the fluidic channel and increase fluidic resistance of the flow of fluid through the fluidic channel. For example, at least a portion of the local constriction may be configured to engage the interior surface of the elastic membrane to divide the fluidic channel into a fluidic channel inlet portion in fluid communication with the inlet and a fluidic channel outlet portion in fluid communication with the outlet. Accordingly, the elastic membrane may be configured to deform and disengage the local constriction to permit fluid flow between the fluidic channel inlet portion and the fluidic channel outlet portion when the average pressure between the inlet and the outlet varies. Moreover, the portion of the local constriction configured to engage the interior surface of the elastic membrane may comprise a circular ridge protruding from an outer surface of the local constriction towards the elastic membrane. The circular ridge may define an inlet chamber of the fluidic channel inlet portion, such that, when the average pressure between the inlet and the outlet varies, fluid within the inlet chamber causes the elastic membrane to deform and disengage the circular ridge to permit fluid flow between the fluidic channel inlet portion and the fluidic channel outlet portion. The ridge may have a height greater than a periphery of the elastic membrane to provide a pre-loaded tension to the elastic membrane. The housing may comprise a circular shape, and an upper portion of the housing may comprise an inverted circular opening configured to expose the outer surface of the elastic membrane to fluid and tissue surrounding the device at the implantation site. The housing, the elastic membrane, and the local constriction may be concentric.

In addition, a first portion of the fluidic channel inlet portion may be defined by a first inner surface of the local constriction, the first portion of the fluidic channel inlet portion fluidically coupling the inlet and the inlet chamber. Additionally, a first portion of the fluidic channel outlet portion may be defined by the outer surface of the local constriction and the interior surface of the elastic membrane, and the first portion of the fluidic channel outlet portion fluidically may be coupled to the outlet via a second portion of the fluidic channel outlet portion defined by a second inner surface of the local constriction. A ratio of an area of the first portion of the fluidic channel outlet portion and an area of the inlet chamber may be selected to create an anti-siphoning effect. The first portion of the fluidic channel inlet portion may be fluidically coupled to the inlet chamber via an opening at a lower portion of the inlet chamber. The outer surface of the local constriction defining the first portion of the fluidic channel outlet portion may comprise a groove extending circumferentially around the circular ridge. Accordingly, the first portion of the fluidic channel outlet portion may be fluidically coupled to the second portion of the fluidic channel outlet portion via an opening within a lower portion of the groove.

In some embodiments, the housing may comprise a tubular shape extending from the inlet to the outlet. The housing may comprise one or more openings configured to expose the outer surface of the elastic membrane to fluid and tissue surrounding the device at the implantation site. A longitudinal length of the one or more openings of the housing may be larger than a longitudinal length of the local constriction. The one or more openings may comprise a first opening at a first portion of the housing, and a second opening at a second portion of the housing opposite the first portion of the housing, and the elastic membrane may comprise a first elastic membrane and a second elastic membrane, the first elastic membrane exposed via the first opening and configured to engage with an upper portion of the local constriction, and the second elastic membrane exposed via the second opening and configured to engage with a lower portion of the local constriction. Accordingly, when the average pressure between the inlet and the outlet varies, the first and second elastic membranes may deform and disengage the upper and lower portions of the local constriction, respectively, to permit fluid flow between the fluidic channel inlet portion and the fluidic channel outlet portion.

Alternatively, the housing may comprise a rectangular shape extending from the inlet to the outlet. An upper portion of the housing may comprise a proximal tapered portion, a distal tapered portion, and an opening between the proximal and distal tapered portions, the opening configured to expose the outer surface of the elastic membrane to fluid and tissue surrounding the device at the implantation site. A longitudinal length of the opening of the housing may be larger than a longitudinal length of the local constriction.

### BRIEF DESCRIPTION OF THE DRAWINGS

FIG. 1A illustrates an exemplary device for the treatment of excess fluid pressure within an eye coupled to a nozzle and a drainage tube, constructed in accordance with some embodiments.

FIG. 1B illustrates the device of FIG. 1A implanted on a scleral surface in accordance with some embodiments.

FIG. 2A is a close up view of the device of FIG. 1A, and FIG. 2B is an exploded view of the components of the device of FIG. 2A.

FIG. 3A illustrates cross-sectional views of the device of FIG. 2A when the valve is in a closed state.

FIG. 3B illustrates cross-sectional views of the device of FIG. 2A when the valve is in a deformed open state in response to distending pressures underneath the elastic membrane, ranging from a high inlet pressure to a low outlet pressure at each end of the device.

FIG. 4 is a graph illustrating exemplary steady state inlet and outlet pressures of the device in vivo.

FIG. 5 illustrates the exemplary device of FIG. 2A coupled to a nozzle and a diffuser plate via a drainage tube in accordance with some embodiments.

FIG. 6A is a schematic diagram illustrating the geometry of another exemplary device for the treatment of excess fluid pressure within an eye in accordance with some embodiments.

FIG. 6B is a cross-sectional view of the exemplary device of FIG. 6A when the valve is in a closed state.

FIG. 6C is a cross-sectional view of the exemplary device of FIG. 6A when the valve is in a deformed open state in response to distending pressures underneath the elastic membrane.

FIG. 7A illustrates yet another exemplary device for the treatment of excess fluid pressure within an eye constructed in accordance with some embodiments.

FIG. 7B is a front view of the device of FIG. 7A, FIG. 7C is a cross-sectional view of the device of FIG. 7A, and FIG. 7D is a perspective cross-sectional view of the device of FIG. 7A.

FIG. 8A is a cross-sectional view of another exemplary device for the treatment of excess fluid pressure within an eye when the valve is in a closed state, constructed in accordance with some embodiments.

FIG. 8B is a cross-sectional view of the exemplary device of FIG. 8A when the valve is in a deformed open state in response to distending pressures underneath the elastic membrane.

FIG. 9A illustrates yet another exemplary device for the treatment of excess fluid pressure within an eye constructed in accordance with some embodiments.

FIG. 9B is a cross-sectional view of the device of FIG. 9A, and FIG. 9C is a cross-sectional view of the device of FIG. 9B.

FIG. 9D is a cross-sectional view of the exemplary device of FIG. 9A when the valve is in a closed state.

FIG. 10A illustrates an exemplary device for the treatment of excess fluid pressure within the brain constructed in accordance with some embodiments.

FIGS. 10B and 10C illustrate cross-sectional views of the device of FIG. 10A.

### DETAILED DESCRIPTION

Systems and methods including an implantable device for treating excessive fluid pressure within a bodily area of a patient, e.g., excessive intraocular pressure within an eye, are provided. In a preferred embodiment, the implantable device includes an elastic membrane defining a fluidic passageway/channel through the device to permit a flow of fluid between an inlet and an outlet of the device, and a local constriction disposed within the fluidic passageway to reduce the area of the fluidic passageway and increase fluidic resistance of the flow of fluid through the passageway. The rate of drainage, and consequently, the IOP, depends on the fluidic resistance of fluidic passageway and the elasticity of the elastic membrane. The elastic membrane is self-regulating to control the flow of fluid, e.g., aqueous humor, from a first bodily area, e.g., an anterior chamber of the eye, through the passageway, to a second bodily area, e.g., a sink outside the eye (e.g., a bleb formed under a scleral flap or the orbital fat space of the eye), and is in direct contact with fluid or tissue surrounding the device at the implantation site, such that the device remains operable regardless of large changes in atmospheric pressure, e.g., due to changes in altitude. The elastic membrane may have a pre-loaded tension and an elasticity that allows the implantable device to maintain intraocular pressures within a desired range, thereby reducing the risk of hypotony in the early post-operative period, i.e., before the bleb has fully formed. Preferably, the local constriction engages with the elastic membrane, such that the pressure distribution between the inlet and outlet of the device must exceed a predetermined threshold to cause the elastic membrane to deform and disengage the local constriction to permit fluid flow through the fluidic passageway.

The devices described herein are expected to provide a number of advantages over the previously-known devices and methods, including: self-regulating pressure within the device to adapt to pressure changes either in the interior chamber of the eye, or distally at the output; limiting the increase of IOP as a result of an increase of downstream pressure due to development of fibrosis; limiting the decrease of IOP to avoid hypotony, especially during the time period shortly following implantation of the implantable device; a low volume design that facilitates implantation on the scleral surface or under a relatively small scleral flap, or even within a diffuser plate; and/or operability regardless of the atmospheric pressure.

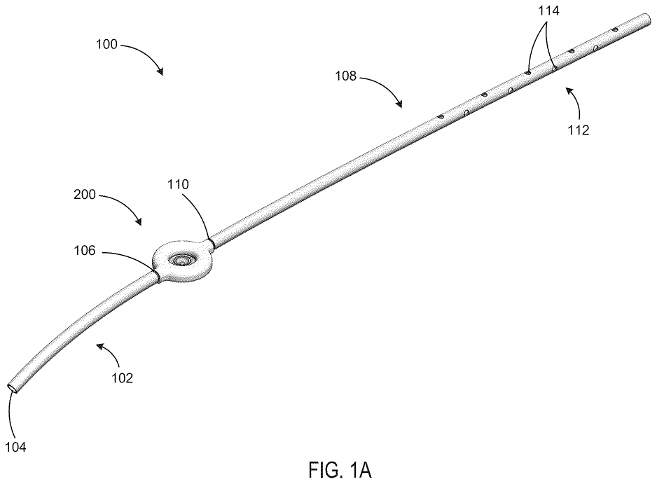

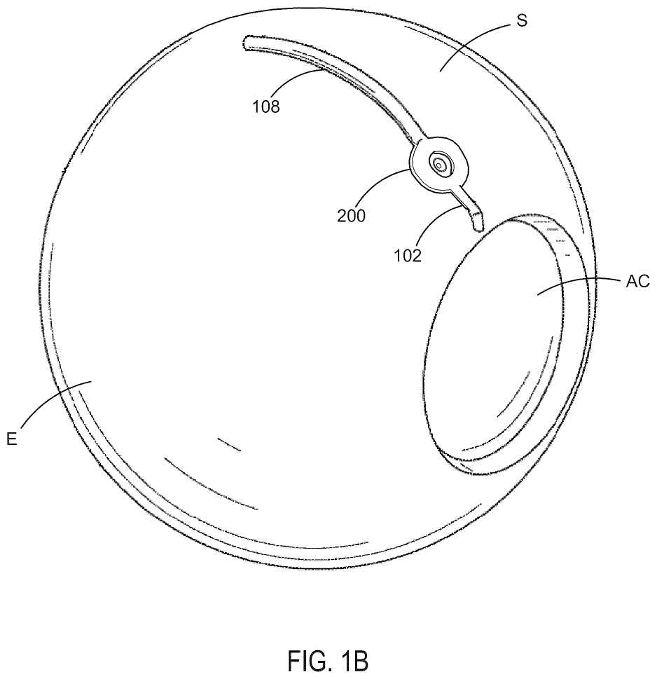

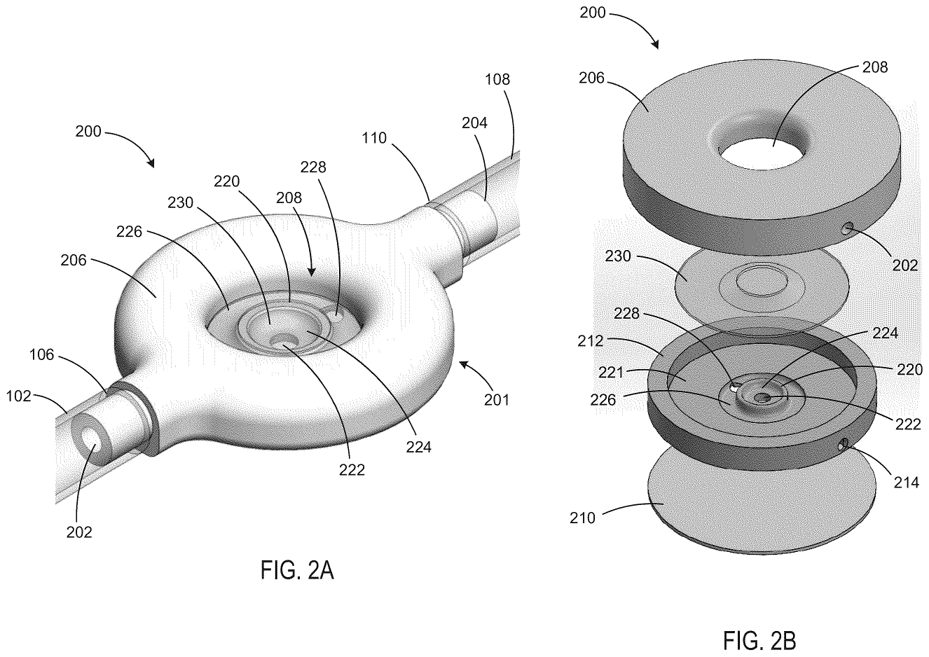

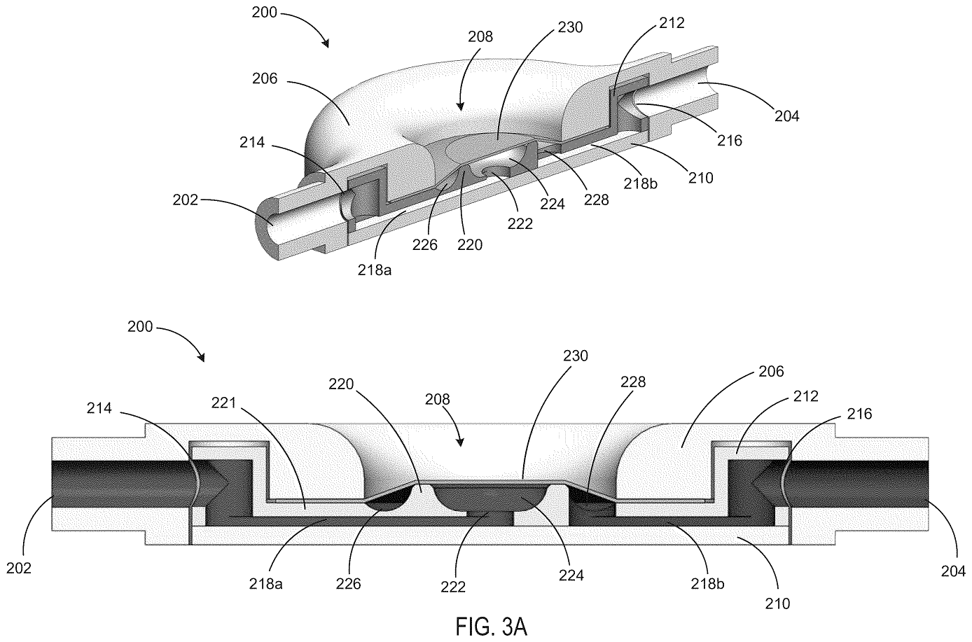

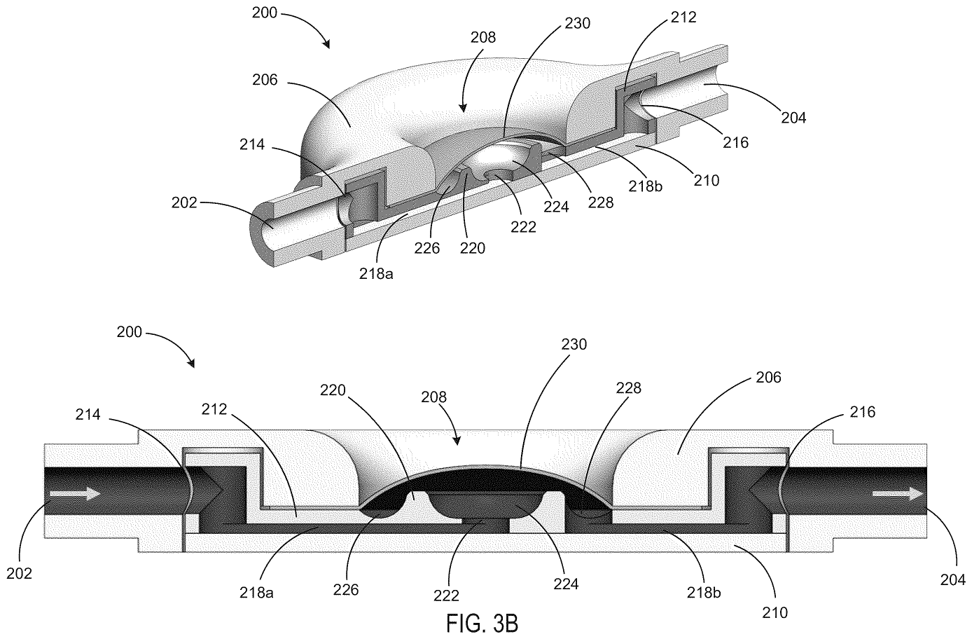

Referring now to FIGS. 1A and 1B, an exemplary system for the treatment of excess intraocular pressure within an eye is described. System 100 includes implantable device 200 for regulating intraocular pressure (IOP) levels within the eye, as described in further detail below with regard to FIGS. 2A to 3B. Device 200 may be coupled to various drainage tubes such that fluid passing through device 200 may be deposited at the orbital fat space of the eye, through a long tube, e.g., drainage tube 108, or on a distal portion of the scleral surface of the patient's eye to thereby treat excess fluid pressure within a patient's eye. For example, as shown in FIG. 1A, device 200 may be coupled to nozzle 102. Nozzle 102 has proximal end 104, distal end 106, and a lumen extending therebetween sized and shaped for flow of aqueous humor from proximal end 104 to distal end 106.

Distal end 106 may be removably coupled to the inlet of device 200, e.g., after implantation of nozzle 102 and after implantation of implantable device 200. Nozzle 102 is designed to extend from the inlet of device 200 and be disposed through the wall of the eye and into the anterior chamber. For example, as shown in FIG. 1B, proximal end 104 of nozzle 102 is sized and shaped to extend through the wall of eye E and into anterior chamber AC when device 200 is implanted beneath the conjunctiva, on the surface of sclera S of eye E, as shown in FIG. 1B. Moreover, a protective patch (not shown), e.g., a layer of allograft tissue, may be positioned above the implantable device to protect the adjacent conjunctival layer from device-induced erosion. To facilitate the introduction of nozzle 102 into the anterior chamber of the eye, proximal end 104 of nozzle 102 may have a conical or sharpened extremity that facilitates piercing of the scleral tissue and introduction of the nozzle into the anterior chamber. Distal end 106 is sized and shaped to receive the inlet of device 200 in a fluid-tight manner such that external bodily fluids cannot enter device 200. Accordingly, the lumen of nozzle 102 is in fluid communication with the fluidic channel of device 200 via the inlet of device 200. Alternatively, in some embodiments, device 200 may be implanted beneath a flap formed in a patient's sclera.

In addition, as shown in FIG. 1A, device 200 may be coupled to drainage tube 108. Drainage tube 108 has proximal end 110, distal region 112, and a lumen extending therebetween sized and shaped for flow of aqueous humor from proximal end 110 to distal region 112. Proximal end 110 may be removably coupled to the outlet of device 200, e.g., after implantation of drainage tube 108 and after implantation of device 200. Proximal end 110 is sized and shaped to receive the outlet of device 200 in a fluid-tight manner such that external bodily fluids cannot enter device 200. Drainage tube 108 preferably has a length such that it extends from the outlet of device 200, and distal region 112 is disposed within a space beneath tissue of the eye, e.g., a space beneath the conjunctiva or an orbital fat space of the eye, for drainage of aqueous humor therein. Distal region 112 may include one or more drainage holes 114 such that the lumen of drainage tube 108 may be in fluid communication with the orbital fat space of the eye. In some embodiments, the distal end of distal region 112 also may include a drainage hole. Drainage tube 108 may be made of, for example, silicone, and may be sufficiently flexible to accommodate the curvature of the patient's eye. Flow exiting through drainage tube 108 is deposited within the distal part of the eye or in the orbital fat space, where it drains primarily to the connecting vein network. Alternatively, flow exiting through drainage tube 108 drains directly to the suprachoroidal space between the sclera and the choroid of the eye.

Referring now to FIGS. 2A and 2B, implantable device 200 for the treatment of excess intraocular pressure within an eye is provided. Implantable device 200 includes housing shell 201, elastic membrane 230, and local constriction 212. Elastic membrane 230 and local constriction 212 may be coupled to and at least partially disposed within housing shell 201, such that at least a portion of elastic membrane 230 is exposed to and in direct contact with fluid and tissue surrounding device 200 at the implantation site. Housing shell 201 includes upper portion 206, lower portion 210, inlet 202, and outlet 204, and may be made from a biocompatible, waterproof or water-resistant plastic such as polyether ether ketone (“PEEK”), polycarbonate, or titanium. The use of PEEK or similar polymer is particularly desirable, as such polymers provide good biocompatibility and long-term structural stability when implanted. Upper portion 206 and lower portion 210 may be separate pieces and molded or glued together during manufacturing of device 200.

Implantable device 200 is configured to be implanted within the eye, e.g., under the conjunctiva, which may be formed using techniques as commonly known in the field of glaucoma filtration surgery. The human eye is generally spherical, having a radius of curvature of approximately 11 mm. Housing shell 201 may include a concave recess on the exterior of lower portion 210 and convex shape on the exterior of upper portion 206, each having a curvature that approximates that of the human eye so that device 200 will lie snugly against the scleral surface beneath the conjunctiva, as shown in FIG. 1B. Preferably, the radius of curvature of lower portion 210 of housing shell 201 is in a range of about 10 mm to about 12 mm, and more preferably about 11 mm.

As shown in FIG. 2A, inlet 202 may be in the form of an inlet connector configured to be removably coupled to various drainage tubes, e.g., distal end 106 of nozzle 102, and outlet 204 may be in the form of an outlet connector configured to be removably coupled to various drainage tubes, e.g., proximal end 110 of drainage tube 108. In some embodiments, the outer surface of the inlet connector and outlet connector may have ridges for a fluid-tight connection with the additional drainage tubes such that external bodily fluids may not enter the fluidic channel of device 200 via inlet 202 and outlet 204. Moreover, inlet 202 and outlet 204 may be disposed on upper portion 206. As shown in FIG. 2B, housing shell 201, elastic membrane 230, and local constriction 212 preferably have circular profiles, such that they are concentric in the assembled configuration where the periphery of elastic membrane 230 is sandwiched between upper portion 206 and local constriction 212. Upper portion 206 may have a toroidal, donut-like shape, including inverted opening 208 sized and shaped to expose at least a portion of elastic membrane 230 therethrough, such that elastic membrane 230 is in direct fluid contact with fluid and tissue surrounding the eye at the implantation site.

As shown in FIG. 2B, local constriction 212 may be disposed on lower portion 210 of housing shell 201, and has inlet end 214 configured to be fluidically coupled to inlet 202 of housing shell 201, and outlet end 216 (FIG. 3A) configured to be fluidically coupled to outlet 204 of housing shell 201. Local constriction 212 is preferably formed of titanium. As will be understood by a person ordinarily skilled in the art, local constriction 212 and lower portion 210 of housing shell 201 may be separate pieces and molded together during manufacturing of device 200, or alternatively, local constriction 212 and lower portion 210 may be formed as a single piece, and accordingly formed of the same material. In addition, local constriction 212 further includes opening 222 in fluid communication with inlet end 214 via a proximal internal fluidic passageway of local constriction 212, and opening 228 in fluid communication with outlet end 216 via a distal internal fluidic passageway of local constriction 212, as described in further detail below with regard to FIGS. 3A and 3B.

As shown in FIG. 2B, local constriction 212 further includes ridge 220 protruding vertically outwardly from an outer surface of local constriction 212, e.g., towards elastic membrane 230 in the assembled configuration. The upper edge of ridge 220 may have a circular profile, thereby defining inlet chamber 224 in fluid communication with inlet end 214 via opening 222. As shown in FIG. 2B, opening 222 may be positioned at the lower portion of inlet chamber 224. Moreover, local constriction 212 may include groove 226 disposed circumferentially around ridge 220. Groove 226 is in fluid communication with outlet end 216 via opening 228. As shown in FIG. 2B, opening 228 may be positioned at the lower portion of groove 228. Preferably, ridge 220 extends to a height above flat portion 221 of local constriction 212, and groove 226 defines a fluidic channel lower than flat portion 221. Accordingly, in the assembled configuration, the periphery of elastic membrane 230 is coupled to and sandwiched between flat portion 221 of local constriction 212 and an inner surface of upper portion 206 of housing shell 201, such that a middle portion of elastic membrane 230 engages ridge 220 and is disposed at a height above the periphery of elastic membrane 230, thereby providing a pre-loaded tension to elastic membrane 230. FIG. 2B illustrates elastic membrane 230 in a deformed configuration having pre-loaded tension due to the force applied thereto by ridge 220, without any additional deformation caused by pressure across inlet 202 and outlet 204; however, as will be understood by a person having ordinary skill in the art, elastic membrane 230 generally has a flat configuration prior to being wrapped over ridge 220. Alternatively, in some embodiments, elastic membrane 230 may have a high elastic modulus, e.g., thickness/stiffness, sufficient to be operable without a pre-loaded tension, such that elastic membrane 230 has an overall flat configuration when there is no pressure acting on the interior surface of elastic membrane 230. For example, in this embodiment, ridge 220 may have the same height as flat portion 221.

Elastic membrane 230 preferably is constructed of a flexible biocompatible material that requires a predetermined level of force to deform, and accordingly, disengage ridge 220, e.g., responsive to pressure fluctuations across inlet 202 and outlet 204. The height of ridge 220 and/or the thickness of elastic membrane 230 may be selected to achieve a target predetermined pressure threshold required to cause elastic membrane 230 to disengage ridge 220, as described in further detail below. Moreover, elastic membrane 230 is leak-proof which ensures that fluid such as, e.g., aqueous humor, or proteinaceous materials contained within the aqueous humor, traveling through the fluidic channel of device 200 does not pass through opening 208 of upper portion 206 of housing shell 201, and similarly, fluid within the eye external to housing shell 201 does not enter the fluidic channel of device 200 via opening 208.

Referring now to FIGS. 3A and 3B, fluid flow across device 200 responsive to pressure across inlet 202 and outlet 204 is provided. FIG. 3A illustrates cross-sectional views of the components of device 200 in the assembled configuration, e.g., when there is no aqueous humor flowing through the device. As shown in FIG. 3A, ridge 220 extends to a height above flat portion 221 of local constriction 212, and groove 226 defines a fluidic channel lower than flat portion 221, such that elastic membrane 230 wrapped over ridge 220 has a pre-loaded tension. Elastic membrane 230 at least partially defines the fluidic channel of device 200 extending within housing shell 201 between inlet 202 and outlet 204, and the addition of local constriction 212 within the fluidic channel reduces the area of the fluid channel and increases fluidic resistance of the flow of fluid through the fluidic channel. As shown in FIG. 3A, the engagement between ridge 220 and the interior surface of elastic membrane 230 divides the fluidic channel into fluidic channel inlet portion 218a (including inlet chamber 224) in fluid communication with inlet 202, and fluidic channel outlet portion 218b (including groove 226) in fluid communication with outlet 204, such that when ridge 220 is engaged with the interior surface of elastic membrane 230, e.g., at low IOP levels (e.g., lower than a threshold value such as in the range of 8 to 10 mmHg), fluid flow may not be permitted between fluidic channel inlet portion 218a and fluidic channel outlet portion 218b. Fluid flow will take place when the average pressure between inlet 202 and outlet 204 varies, e.g., exceeds a predetermined threshold, thereby causing elastic membrane 230 to deform sufficiently to disengage ridge 220.

In this case, the balance of forces between the atmospheric pressure forces applied against the exterior surface of elastic membrane 230, e.g., through inverted opening 208, and pressure forces applied against the interior surface of elastic membrane 230 as a result of the flow of aqueous humor from the anterior chamber of the eye through inlet 202 and fluidic channel inlet portion 218a into inlet chamber 224, define the geometry of elastic membrane 230, and accordingly, the size of the gap formed between the interior surface of elastic membrane 230 and the upper edge of ridge 220 upon deformation of elastic membrane 130, and therefore the hydraulic resistance of the fluidic channel of device 200. If IOP increases and/or if distal pressure increases, e.g., due to progressive fibrosis formation at outlet 204, the pressure acting the interior surface of elastic membrane 230 will increase, thereby causing elastic membrane 230 to deform even further and decreasing fluidic resistance to flow, which in turn will lower upstream pressures, restoring IOP levels to initial values. Accordingly, device 200 will maintain a near-constant desired IOP at physiological levels at inlet end 202 even if flow or pressure at outlet 204 changes.

As shown in FIG. 3A, local constriction 212 may be configured such that an inner surface of the proximal region of local constriction 212 and lower portion 210 of housing shell 201 define a first portion of fluidic channel inlet portion 218a extending between inlet end 214 fluidically coupled to inlet 202, and opening 222 of inlet chamber 224. The second portion of fluidic channel inlet portion 218a, e.g., inlet chamber 224 of fluidic channel inlet portion 218a defined by ridge 220 and the interior surface of elastic membrane 230, is in fluid communication with outlet end 214 via opening 222, such that fluid may flow through inlet end 214, across the first portion of fluidic channel inlet portion 218a, and through opening 222 into inlet chamber 224. Fluid may be maintained within inlet chamber 224 until the average pressure between inlet 202 and outlet 204 exceeds a predetermined threshold, such that the fluid within inlet chamber 224 applies an upward force against the interior surface of elastic membrane 230, thereby causing elastic membrane 230 to deform and disengage ridge 220 and fluidically couple fluidic channel inlet portion 218a and fluidic channel outlet portion 218b, as described above.

Moreover, as shown in FIG. 3A, local constriction 212 may be configured such that groove 226 and the interior surface of elastic membrane 230 define a first portion of fluidic channel outlet portion 218b, and an inner surface of the distal region of local constriction 212 and lower portion 210 of housing shell 201 define a second portion of fluidic channel inlet portion 218b extending between opening 228 of groove 226 and outlet end 216 fluidically coupled to outlet 204. Accordingly, groove 226 is in fluid communication with outlet end 216 via opening 228, such that when the average pressure between inlet 202 and outlet 204 exceeds the predetermined threshold, thereby causing elastic membrane 230 to deform and disengage ridge 220, fluid may flow from inlet chamber 224 over ridge 220 into groove 226, and from groove 226 through opening 228, across the second portion of fluidic channel outlet portion 218b, and exit via outlet end 216, as shown in FIG. 3B.

As described above, fluid flow from inlet 202 to inlet chamber 224 of fluidic channel inlet portion 218a applies pressure to the interior surface of elastic membrane 230. If the internal pressure within fluidic channel inlet portion 218a increases beyond a predetermined amount, elastic membrane 230 will “bulge” out, e.g., deform within opening 208 and disengage ridge 220 as shown in FIG. 3B, thereby decreasing the hydraulic resistance of the fluidic channel of device 200, and fluidically couple fluidic channel inlet portion 218a and fluidic channel outlet portion 218b to permit fluid to flow from inlet chamber 224 to groove 226.

Here, a simple viscous resistance law is applied:

$$\frac{{P}_{i n} - {P}_{o u t}}{R} = Q ; therefore , {P}_{i n} = {P}_{out} + R * Q$$

As downstream external pressure (Pₒᵤₜ) increases, the pressure within fluidic channel inlet portion 218a will initially increase as it is always bounded between upstream intraocular pressure (Pᵢₙ) at inlet 202 to downstream external pressure (Pₒᵤₜ) at outlet 204. Thus, elastic membrane 230 will “bulge” into opening 208. This will increase the cross-sectional area and overall volume of the fluidic channel of device 200, and thus lower hydraulic resistance (R) to a level such that Pᵢₙ will be effectively unchanged.

FIG. 3B illustrates expected operation of implantable device 200 when there is, for example, an increase in IOP levels at inlet 202. For example, if flow increases, IOP will increase and the average pressure forces within fluidic channel inlet portion 218a will increase, which will cause elastic membrane 230 to bulge, e.g., deform into opening 208 and disengage ridge 220 when the average pressure forces within fluidic channel inlet portion 218a exceeds the predetermined threshold, resulting in fluid communication between fluidic channel inlet portion 218a and fluidic channel outlet portion 218b, a larger flow area, smaller fluidic resistance, and consequently a decrease in IOP, thereby allowing the IOP to be maintained at a pre-determined desired level. Any scenario causing IOP to increase will result in deformable structure equilibrating at a new, larger flow area and increased flow that will result in turn reduce IOP. As shown in FIG. 3B, the expansion of elastic membrane 230 has a parabolic shape along elastic membrane 230. Similarly, if pressure at outlet 204 increases, for example, due to the development of fibrosis at outlet 204, the average pressure within fluidic channel outlet portion 218b will increase, causing elastic membrane 230 to deform into opening 208 and disengage ridge 220, which in turn will result in increased flow area and smaller fluidic resistance within the fluidic channel of device 200. Consequently, the increase of IOP at inlet 202 will be limited.

In accordance with one aspect, if IOP at inlet 202 decreases, for example, during the period of time right after implantation which may cause hypotony, the average pressure within fluidic channel inlet portion 218a will decrease, which may cause elastic membrane 230 to deform toward inlet chamber 224, thereby strengthening the seal between elastic membrane 230 and ridge 220 and increasing fluidic resistance within the fluidic channel of device 200, and further preventing fluid flow from fluidic channel inlet portion 218a to fluidic channel outlet portion 218b. This in turn will limit the decrease of IOP at inlet 202 and reduce the risk of hypotony.

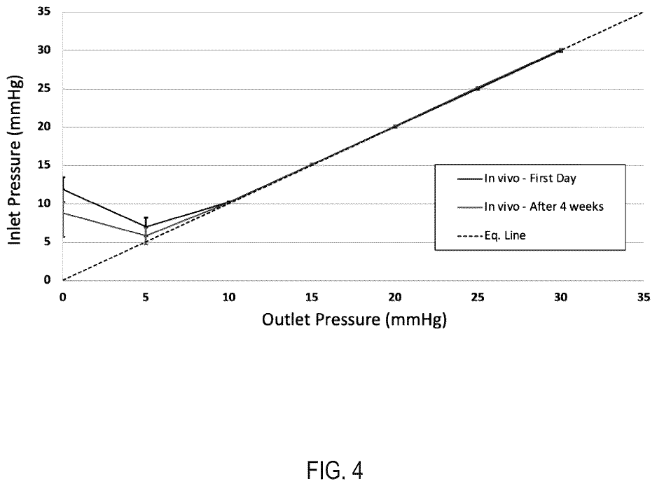

For a given flow through the fluidic channel of device 200, device 200 becomes an upstream pressure regulator in the sense that, if fluidic pressure within fluidic channel inlet portion 218a increases, elastic membrane 230 will deform into opening 208 and the hydraulic resistance will decrease. In this case, the pressure at inlet 202 may be maintained relatively constant. FIG. 4 is a graph depicting upstream/inlet pressure at the inlet end of the device, i.e., internal intraocular pressure within the eye, versus downstream/outlet pressure at the outlet end of the device, external pressure, from an in vivo test with a prototype of device 200 on the first day following implantation and at four weeks after implantation. As shown in FIG. 4, when downstream/outlet pressure varies from 0 to 10 mmHg, upstream/inlet pressure may initially decrease slightly and then begin to slightly increase. Having resistance when downstream/outlet pressure is low is desirable to prevent hypotony; however, resistance is ideally zero when downstream/outlet pressure is high. The V-shape decrease/increase of upstream/inlet pressure at low downstream/outlet pressure levels may be flattened, e.g., upstream/inlet pressure may be maintained relatively constant, by adjusting one or more parameters of device 200, e.g., the height of ridge 220, etc.

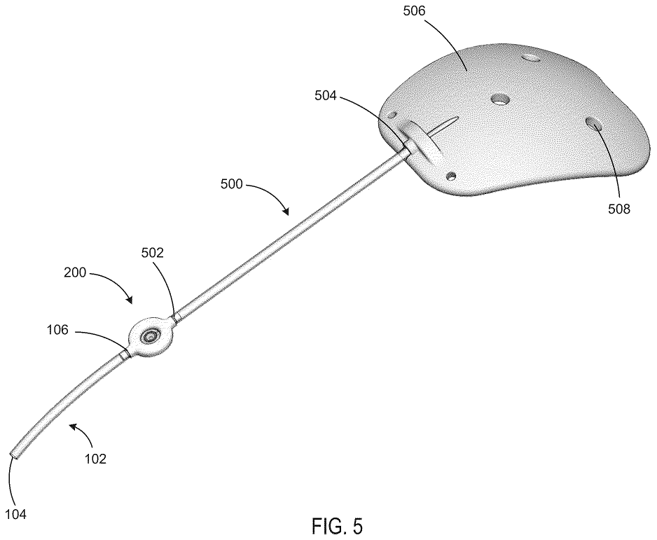

Referring now to FIG. 5, device 200 may be coupled to distal end 106 of nozzle 102 via inlet 202 as described above, and further coupled to diffuser plate 506, e.g., a Seton tube, via drainage tube 500. For example, outlet 204 of device 200 may be coupled to proximal end 502 of drainage tube 500, and distal region 504 of drainage tube 500 may be fluidically coupled to diffuser plate 506. For example, diffuser plate 506 may include a groove shaped and sized to receive drainage tube 500, and drainage tube 500 may be maintained within the groove via, e.g., friction or an adhesive. Flow exiting outlet 204 travels into proximal end 502 and through drainage tube 500 into diffuser plate 506 which is sutured on the episcleral surface in the back of the eye. Alternatively, as described above with reference to FIG. 1A, device 200 may be connected to drainage tube 108 disposed in a space, e.g., the orbital fat space, of the eye, such that aqueous humor may be absorbed into the orbital fat space of the eye. Diffuser plate 506 may be curved to accommodate the curvature of the eye and may include eyelets 508 shaped and sized to permit diffuser plate 506 to be implanted and remain in position once implanted on an exterior surface of the eye via, e.g., sutures. In this embodiment, diffuser plate 506 may include one or more drainage holes along its upper surface such that the lumen of drainage tube 500 may be in communication with the upper surface of diffuser plate 506.

In accordance with another aspect, device 200 may be designed to be implanted within the diffuser plate on the scleral surface of a human eye. In this embodiment, the nozzle is sized and shaped to extend from within the diffuser plate along the curvature of the eye and to be disposed through the wall of the eye and into the anterior chamber. Flow enters the implantable device through the nozzle coupled to the inlet of the device and exits through the outlet into the diffuser plate and is ultimately deposited beneath the tissue of the eye, where it drains primarily to the connecting vein network.

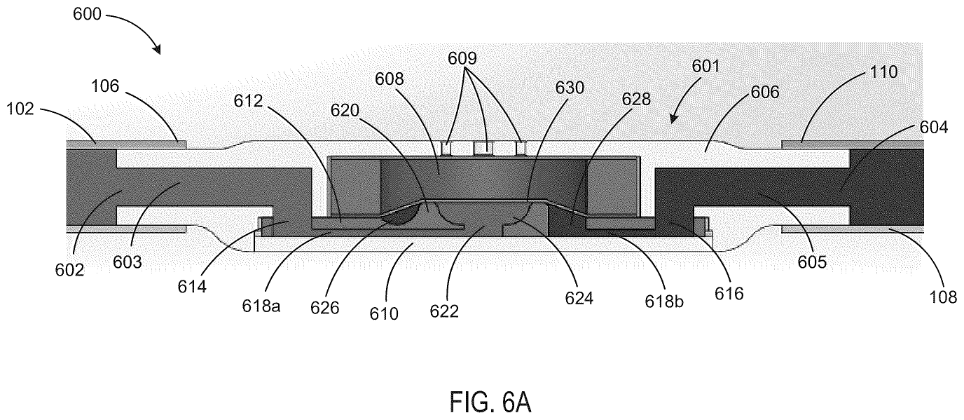

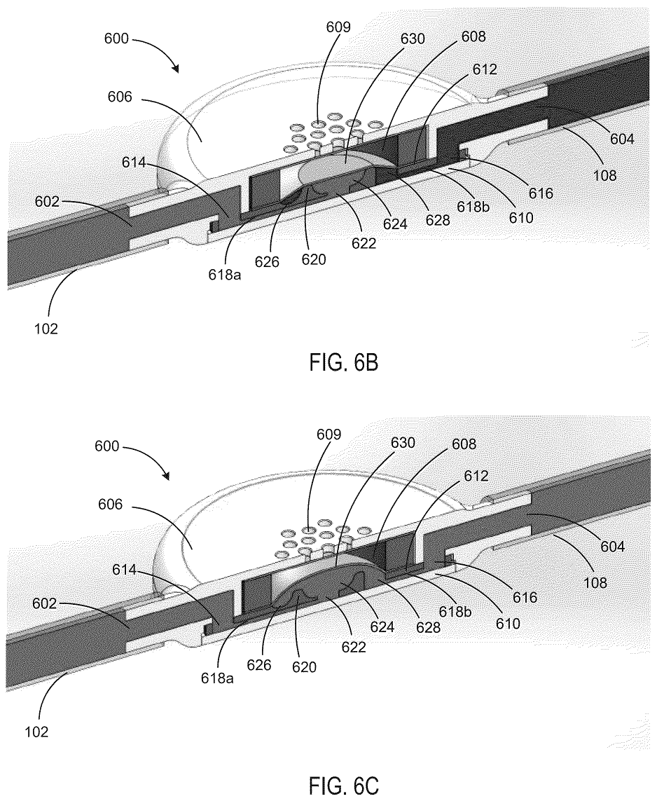

Referring now to FIGS. 6A to 6C, another exemplary device for the treatment of excess intraocular pressure within an eye is provided. Implantable device 600 may be constructed similar to implantable device 200. For example, inlet 602, outlet 604, and lower portion 610 of housing shell 601 correspond with inlet 202, outlet 204, and lower portion 210 of housing shell 201, local constriction 612 having inlet end 614, outlet end 616, openings 622, 628, circular ridge 620 defining inlet chamber 624 of fluidic channel inlet portion 618a, and groove 626 defining a first portion of fluidic channel outlet portion 618b correspond with local constriction 212 having inlet end 214, outlet end 216, openings 222, 228, circular ridge 220 defining inlet chamber 224 of fluidic channel inlet portion 218a, and groove 226 defining the first portion of fluidic channel outlet portion 218b, and elastic membrane 630 correspond with elastic membrane 230. Accordingly, inlet 602 is configured to be fluidically coupled to a nozzle, e.g., distal end 106 of nozzle 102, and outlet 604 is configured to be fluidically coupled to a drainage tube, e.g., proximal end 110 of drainage tube 108. Device 600 differs from device 200 in that upper portion 606 of housing shell 601 includes non-hermetic, enclosed cavity 608, such that in an assembled configuration, elastic membrane 630 is disposed within cavity 608. As shown in FIG. 6A, upper portion 606 further includes one or more holes 609 sized and shaped to permit vapor to pass therethrough, e.g., even if holes 609 are covered by tissue ingrowth, such that the outer surface of elastic membrane 630 is in direct fluid contact with fluid and tissue surrounding device 600 at the implantation site.

FIG. 6B depicts expected operation of device 600 at low IOP levels, e.g., when there is no flow of aqueous humor through device 600, and FIG. 6C depicts expected operation of device 600 when the average pressure between inlet 602 and outlet 604 varies, e.g., if IOP increases and/or if distal pressure increases beyond a predetermined threshold, such that fluid within fluid channel inlet portion 618a causes elastic membrane 620 to deform and disengage from ridge 620 to permit fluid flow between fluid channel inlet portion 618a and fluid channel outlet portion 618b, e.g., through the gap formed between the interior surface of elastic membrane 630 and the upper edge of ridge 620.

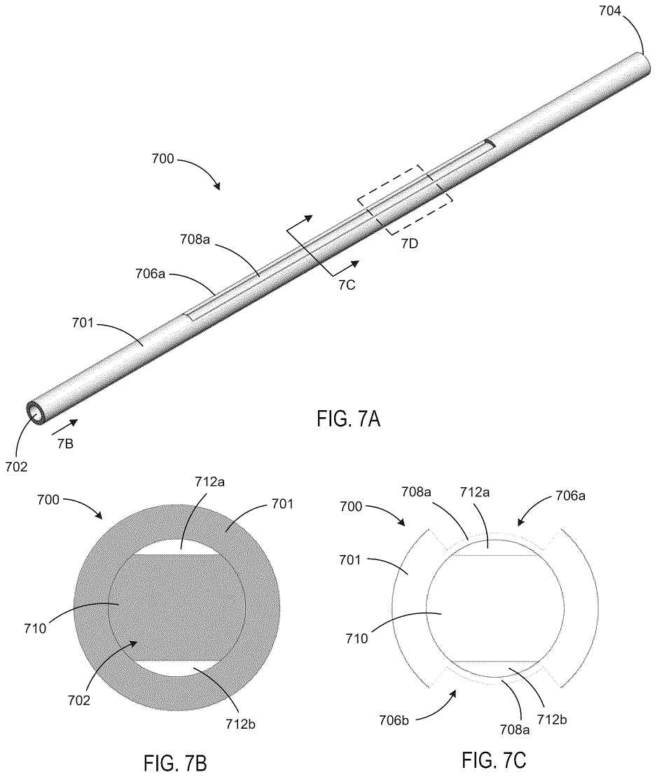

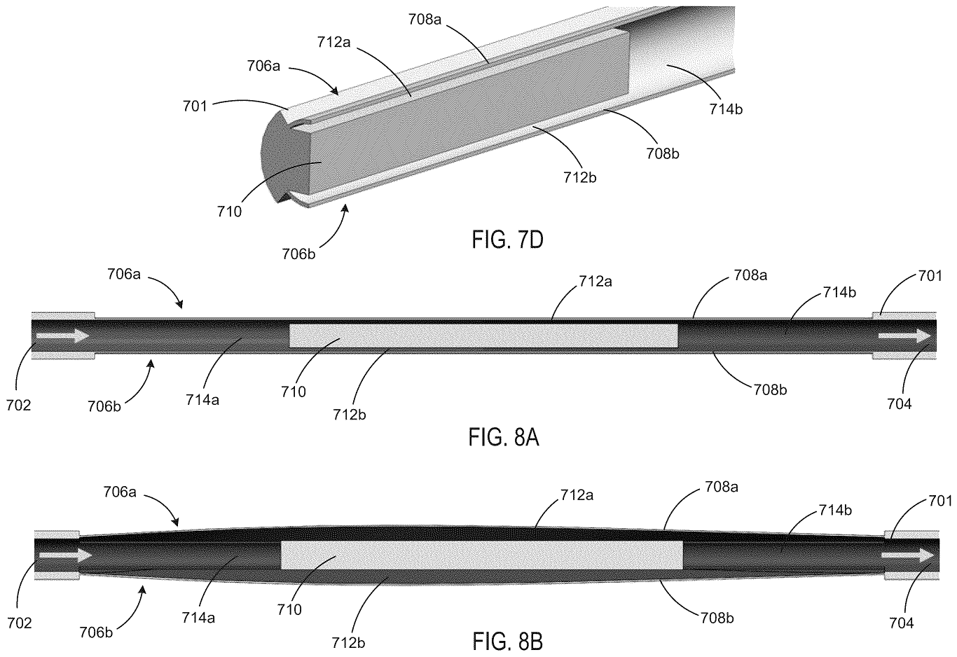

Referring now to FIGS. 7A to 7D, another exemplary device for the treatment of excess intraocular pressure within an eye is provided. Implantable device 700 includes housing shell 701 having a tubular shape defining inlet 702, outlet 704, and a fluidic passageway/channel extending between inlet 702 and outlet 704. Inlet 702 may be in the form of an inlet connector configured to be removably coupled to various drainage tubes, e.g., distal end 106 of nozzle 102, and outlet 704 may be in the form of an outlet connector configured to be removably coupled to various drainage tubes, e.g., proximal end 110 of drainage tube 108. Alternatively, like outlet 204 of device 200, outlet 704 may be fluidically coupled to, e.g., a diffuser plate, and/or device 700 may be disposed within a diffuser plate, as described above with regard to FIG. 5. In some embodiments, the outer surface of the inlet connector and outlet connector may have ridges for a fluid-tight connection with the additional drainage tubes such that external bodily fluids may not enter the fluidic channel of device 700 via inlet 702 and outlet 704.

As shown in FIG. 7A, housing shell 701 may include an opening, e.g., upper opening 706a, sized and shaped to expose an elastic membrane, e.g., upper elastic membrane 708a, such that upper elastic membrane 708a is in direct contact with fluid and tissue surrounding device 700 at the implantation site. Moreover, as shown in FIGS. 7C and 7D, housing shell 701 may include another opening, e.g., lower opening 706b, opposite upper opening 706a and sized and shaped to expose another elastic membrane, e.g., lower elastic membrane 708b, such that lower elastic membrane 708b is in direct contact with fluid and tissue surrounding device 700 at the implantation site.

As shown in FIG. 7B, which is a front view of device 700, device 700 includes local constriction 710 disposed within at least a portion of the fluidic passageway of housing shell 701 to reduce the area of the fluidic passageway and increase fluidic resistance of the flow of fluid through the passageway. In addition, local constriction 710 may be sized and shaped to define upper fluidic channel 712a between the upper surface of local constriction 710 and the interior surface of upper elastic membrane 708a, and lower fluidic channel 712b between the lower surface of local constriction 710 and the interior surface of lower elastic membrane 708b, such that fluid flowing through the fluidic channel of device 700 may flow across local constriction 710 via either upper fluidic channel 712a or lower fluidic channel 712b. Local constriction 710 and housing shell 701 may be separate pieces and molded together during manufacturing of device 700, or alternatively, local constriction 710 and housing shell 701 may be formed as a single piece, and accordingly formed of the same material.

Referring now to FIGS. 8A and 8B, fluid flow across device 700 responsive to pressure fluctuations across inlet 702 and outlet 704 is provided. FIG. 8A illustrates cross-sectional views of the components of device 700 in the assembled configuration, depicting expected operation of device 700 at regular IOP levels, e.g., when the downstream external pressure is very low (<3 mmHg). As shown in FIG. 8A, local constriction 710 may have a rectangular cross-sectional area, such that the upper surface of local constriction 710 engages with the interior surface of upper elastic membrane 708a, and the lower surface of local constriction 710 engages with the interior surface of lower elastic membrane 708b when upper elastic membrane 708a and lower elastic membrane 708b are in their undeformed states, e.g., at regular IOP levels. Accordingly, local constriction 710 divides the fluidic channel of device 700 into fluidic channel inlet portion 714a and fluidic channel outlet portion 714b, such that fluidic channel inlet portion 714a is not in fluid communication with fluidic channel outlet portion 714b when upper elastic membrane 708a and lower elastic membrane 708b are in their undeformed states and engage with local constriction 710.

FIG. 8B illustrates expected operation of implantable device 700 when there is, for example, an increase in IOP levels at inlet 702. For example, if flow increases, IOP will increase and the average pressure forces within fluidic channel inlet portion 714a will increase, which will cause upper elastic membrane 708a and lower elastic membrane 708b to bulge, e.g., deform into upper opening 706a and lower opening 706b, respectively, and disengage local constriction 710 when the average pressure forces within fluidic channel inlet portion 714a exceeds the predetermined threshold, resulting in fluid communication between fluidic channel inlet portion 714a and fluidic channel outlet portion 714b, a larger flow area, smaller fluidic resistance, and consequently a decrease in IOP, thereby allowing the IOP to be maintained at a pre-determined desired level. Any scenario causing IOP to increase will result in deformable structure equilibrating at a new, larger flow area and increased flow that will result in turn reduce IOP. As shown in FIG. 8B, the expansion of upper elastic membrane 708a and lower elastic membrane 708b has a parabolic shape along upper elastic membrane 708a and lower elastic membrane 708b, respectively. Similarly, if pressure at outlet 704 increases, for example, due to the development of fibrosis at outlet 704, the average pressure within fluidic channel outlet portion 714b will increase, causing upper elastic membrane 708a and lower elastic membrane 708b to deform into upper opening 706a and lower opening 706b, respectively, and disengage local constriction 710, which in turn will result in increased flow area and smaller fluidic resistance within the fluidic channel of device 700. Consequently, the increase of IOP at inlet 702 will be limited.

In accordance with one aspect, if IOP at inlet 702 decreases, for example, during the period of time right after implantation which may cause hypotony, the average pressure within fluidic channel inlet portion 714a will decrease, which may cause upper elastic membrane 708a and lower elastic membrane 708b to deform toward local constriction 710, thereby strengthening the seal between local constriction 710 and upper elastic membrane 708a and lower elastic membrane 708b, and increasing fluidic resistance within the fluidic channel of device 700, and further preventing fluid flow from fluidic channel inlet portion 714a to fluidic channel outlet portion 714b. This in turn will limit the decrease of IOP at inlet 702 and reduce the risk of hypotony. For a given flow through the fluidic channel of device 700, device 700 becomes an upstream pressure regulator in the sense that, if fluidic pressure within fluidic channel inlet portion 714a increases, upper elastic membrane 708a and lower elastic membrane 708b will deform into upper opening 706a and lower opening 706b, respectively, and the hydraulic resistance will decrease. In this case, the pressure at inlet 702 may be maintained relatively constant.

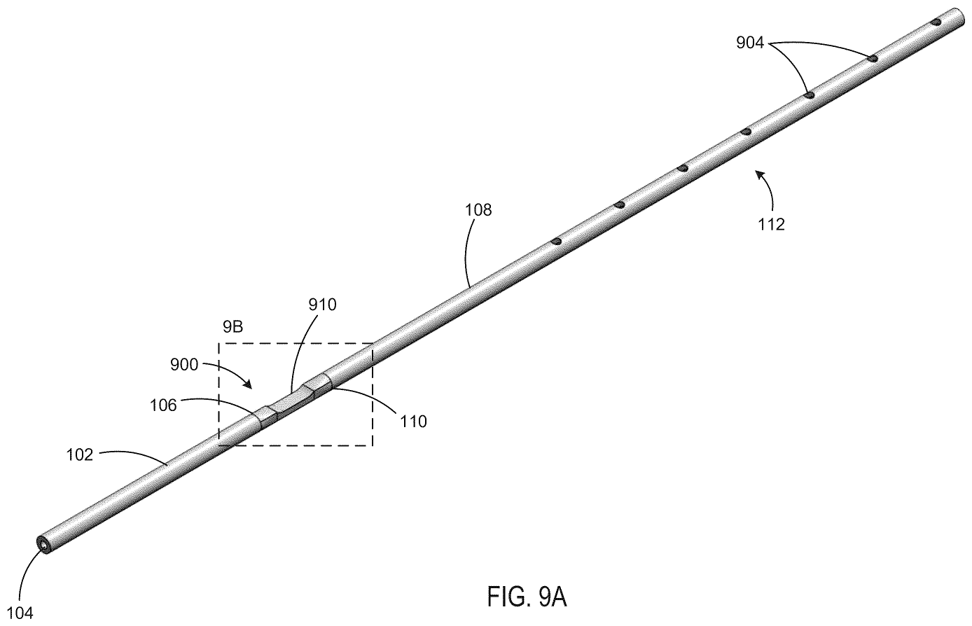

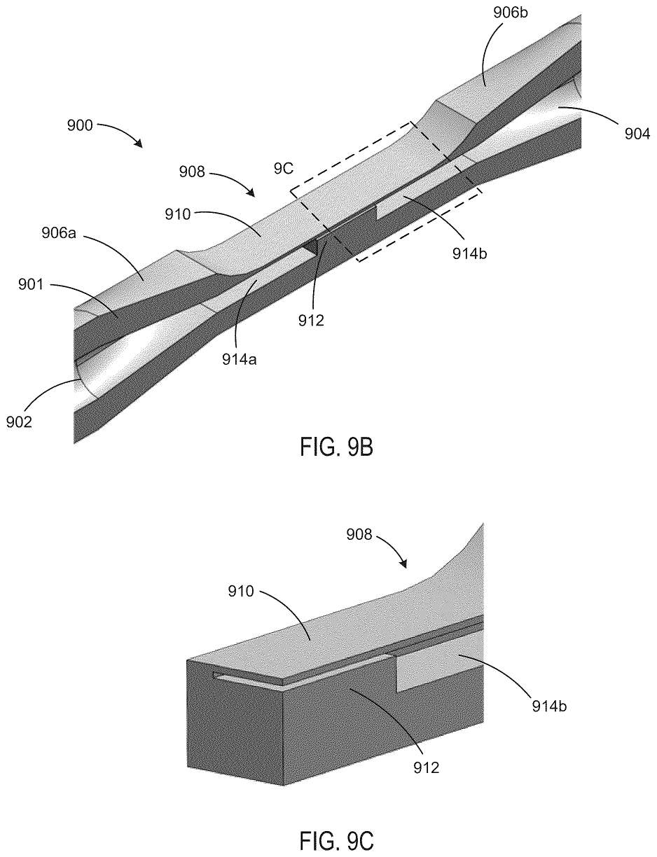

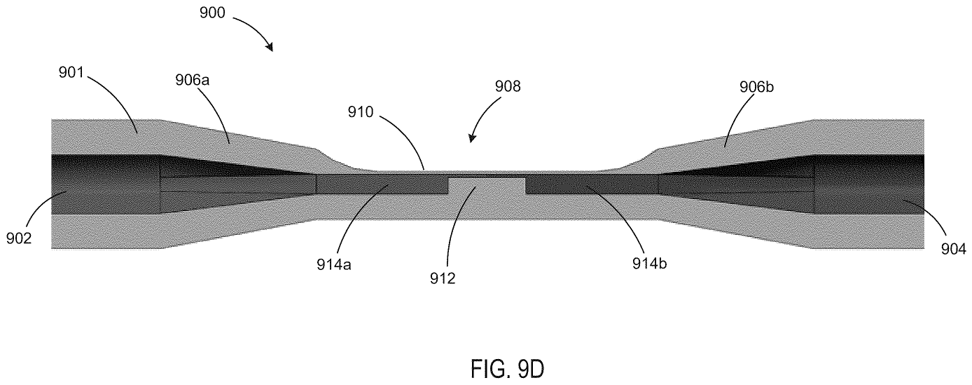

Referring now to FIGS. 9A to 9D, another exemplary device for the treatment of excess intraocular pressure within an eye is provided. Like device 200 and device 700, device 900 may be coupled to various drainage tubes such that fluid passing through device 900 may be deposited with an orbital fat space or on the sclera of the patient's eye to thereby treat excess fluid pressure within a patient's eye. For example, as shown in FIG. 9A, an inlet of device 900 may be fluidically coupled to distal end 106 of nozzle 102, and an outlet of device 900 may be fluidically coupled to proximal end 110 of drainage tube 108 having one or more drainage holes 114 at distal region 112 of drainage tube 108. Alternatively, like outlet 204 of device 200, the outlet of device 900 may be fluidically coupled to, e.g., a diffuser plate, and/or device 900 may be disposed within a diffuser plate, as described above with regard to FIG. 5. In some embodiments, the outer surface of the inlet connector and outlet connector may have ridges for a fluid-tight connection with the additional drainage tubes such that external bodily fluids may not enter the fluidic channel of device 900 via the inlet or outlet of device 900.

As shown in FIG. 9B, implantable device 900 includes housing shell 901 having a tubular shape defining inlet 902, outlet 904, and a fluidic passageway/channel extending between inlet 902 and outlet 904. Inlet 902 may be in the form of an inlet connector configured to be removably coupled to various drainage tubes, e.g., distal end 106 of nozzle 102, and outlet 904 may be in the form of an outlet connector configured to be removably coupled to various drainage tubes, e.g., proximal end 110 of drainage tube 108. In addition, housing shell 901 may include proximal tapered portion 906a, distal tapered portion 906b, and opening 908 disposed between proximal tapered portion 906a and distal tapered portion 906b. For example, the cross-sectional area of housing shell 901 may decrease along proximal tapered portion 906a and along distal tapered portion 906b in the direction towards opening 908. Opening 908 is sized and shaped to expose elastic membrane 910 therethrough, such that elastic membrane 910 is in direct contact with fluid and tissue surrounding device 900 at the implantation site.

As shown in FIGS. 9B and 9C, device 900 includes local constriction 912 disposed within at least a portion of the fluidic passageway of housing shell 901 to reduce the area of the fluidic passageway and increase fluidic resistance of the flow of fluid through the passageway. Local constriction 912 may have a rectangular cross-sectional area, such that the upper surface of local constriction 912 engages with the interior surface of elastic membrane 910 when elastic membrane 910 is in its undeformed state, e.g., at regular IOP levels. Accordingly, local constriction 912 divides the fluidic channel of device 900 into fluidic channel inlet portion 914a and fluidic channel outlet portion 914b, such that fluidic channel inlet portion 914a is not in fluid communication with fluidic channel outlet portion 914b when elastic membrane 910 is in its undeformed states and engages with local constriction 910. Local constriction 912 and housing shell 901 may be separate pieces and molded together during manufacturing of device 900, or alternatively, local constriction 912 and housing shell 901 may be formed as a single piece, and accordingly formed of the same material.

FIG. 9D is a cross-sectional view of the components of device 900 in the assembled configuration, depicting expected operation of device 900 at regular IOP levels, e.g., when the downstream external pressure is very low (<3 mmHg). In accordance with some aspects, when there is an increase in IOP levels at inlet 902, the average pressure forces within fluidic channel inlet portion 914a will increase, which will cause elastic membrane 910 to bulge, e.g., deform into opening 908 and disengage local constriction 912 when the average pressure forces within fluidic channel inlet portion 914a exceeds the predetermined threshold, resulting in fluid communication between fluidic channel inlet portion 914a and fluidic channel outlet portion 914b, a larger flow area, smaller fluidic resistance, and consequently a decrease in IOP, thereby allowing the IOP to be maintained at a pre-determined desired level. Any scenario causing IOP to increase will result in deformable structure equilibrating at a new, larger flow area and increased flow that will result in turn reduce IOP. As described above, the expansion of elastic membrane 910 may have a parabolic shape along elastic membrane 910. Similarly, if pressure at outlet 904 increases, for example, due to the development of fibrosis at outlet 904, the average pressure within fluidic channel outlet portion 914b will increase, causing elastic membrane 910 to deform into opening 908 and disengage local constriction 912, which in turn will result in increased flow area and smaller fluidic resistance within the fluidic channel of device 900. Consequently, the increase of IOP at inlet 902 will be limited.

In accordance with one aspect, if IOP at inlet 902 decreases, for example, during the period of time right after implantation which may cause hypotony, the average pressure within fluidic channel inlet portion 914a will decrease, which may cause elastic membrane 910 to deform toward local constriction 912, thereby strengthening the seal between local constriction 912 and elastic membrane 910, and increasing fluidic resistance within the fluidic channel of device 900, and further preventing fluid flow from fluidic channel inlet portion 914a to fluidic channel outlet portion 914b. This in turn will limit the decrease of IOP at inlet 902 and reduce the risk of hypotony. For a given flow through the fluidic channel of device 900, device 900 becomes an upstream pressure regulator in the sense that, if fluidic pressure within fluidic channel inlet portion 914a increases, elastic membrane 910 will deform into opening 908 and the hydraulic resistance will decrease. In this case, the pressure at inlet 902 may be maintained relatively constant.

The implantable devices described herein may be configured for implantation at an implantation site within the patient, such that the inlet of the device is in fluid communication with a brain ventricle of the patient for draining cerebrospinal fluid (CSF) from the brain ventricle for the treatment of hydrocephalus. Accordingly, the elastic membrane may be self-regulating to control the flow of CSF from the brain ventricle through the passageway to another bodily area, e.g., for natural absorption and/or disposal by the patient's body, regardless of large changes in atmospheric pressure, e.g., due to changes in altitude.

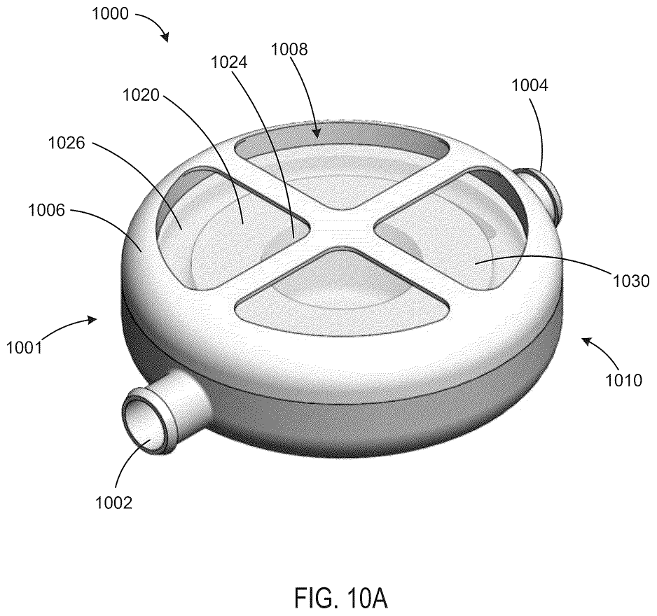

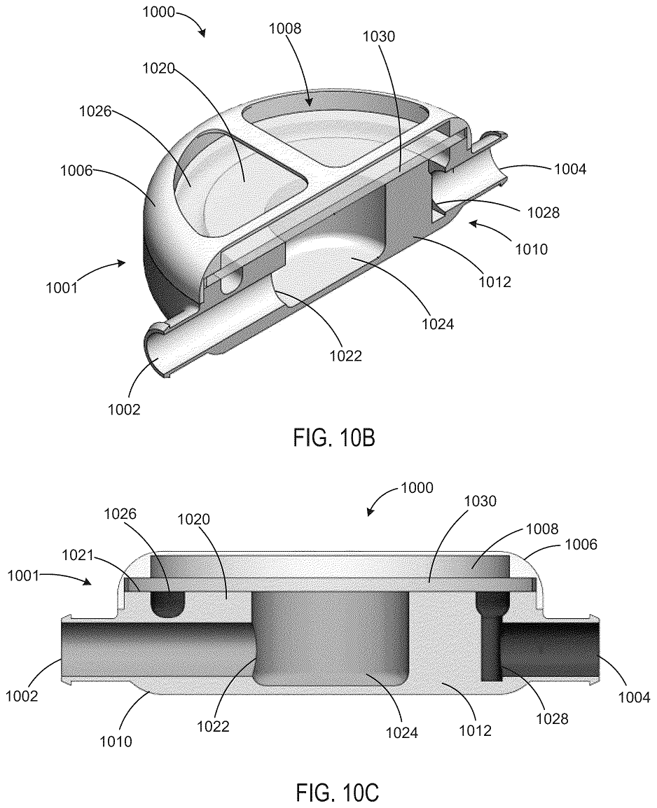

Referring now to FIGS. 10A to 10C, an exemplary device for the treatment of excess fluid pressure within the brain, e.g., to treat hydrocephalus, is provided. Implantable device 1000 may be constructed similar to implantable device 200. For example, device 1000 may include housing shell 1001 having upper portion 1006 including one or more openings 1008, lower portion 1010, inlet 1002, and outlet 1004, local constriction 1012 having ridge 1020 protruding vertically outwardly from an outer surface of local constriction 1012 and defining inlet chamber 1024 having opening 1022 in fluid communication with inlet 1002 via a proximal internal fluidic passageway of local constriction 1012 and groove 1026 disposed circumferentially around ridge 1020 and having opening 1028 in fluid communication with outlet 1004 via a distal internal fluidic passageway of local constriction 1012, and elastic membrane 1030 coupled to housing shell 1001 along its periphery such its interior surface is releasably engageable with ridge 1020 (e.g., when an average pressure between the inlet and the outlet varies) and its exterior surface is exposed to and in direct contact with fluid and tissue surrounding device 1000 at the implantation site, which correspond with housing shell 201 having upper portion 206 including opening 208, lower portion 210, inlet 202, and outlet 204, local constriction 212 having ridge 220 and defining inlet chamber 224 having opening 222 in fluid communication with inlet 202 and groove 226 having opening 228 in fluid communication with outlet 204, and elastic membrane 230. As will be understood by a person having ordinary skill in the art, while FIG. 10A illustrates upper portion 1006 having four openings 1008, upper portion 1006 may have less or more than four openings, each configured to expose the exterior surface of elastic membrane 1030 to fluid and tissue surrounding device 1000.

Upper portion 1006, lower portion 1010, and local constriction 1012 may be made of a biocompatible, waterproof or water-resistant, long-term implantable material, e.g., PEEK, polycarbonate, or titanium, and elastic membrane 1030 may be constructed from a highly elastic, long-term implantable material, e.g., silicone. Local constriction 1012 and lower portion 1010 of housing shell 1001 may be formed as a single piece, and accordingly formed of the same material, or alternatively, local constriction 1012 and lower portion 1010 may be separate pieces and molded together during manufacturing of device 1000. Additionally, upper portion 1006 and lower portion 1010 may be separate pieces and molded or glued together during manufacturing of device 1000. Housing shell 1001 may have an overall diameter of between 3 to 12 mm, and preferably 6 mm, and an overall thickness of between 1 and 3 mm, and preferably 1.5 mm. Thus, device 1000 may be about three times smaller than a Codman Hakim Programmable Valve in all dimensions, and about 30 times smaller in volume.

Inlet 1002 may be configured to be removably and fluidically coupled to a nozzle in a fluid-tight manner such that external bodily fluids cannot enter device 1000, the nozzle configured to extend from device 1000 and having an inlet end disposed in fluidic communication with a source of CSF, e.g., a ventricle of the brain. Outlet 1004 may be configured to be removably and fluidically coupled to a drainage tube in a fluid-tight manner such that external bodily fluids cannot enter device 1000, the drainage tube having a length selected such that an outlet end of the drainage tube extends from device 1000 and is disposed in fluidic communication with another part of the body, e.g., the peritoneal cavity. Accordingly, when an average pressure between inlet 1002 and outlet 1004 varies, CSF may flow from the brain ventricles through the nozzle into device 1000 via inlet 1002, and through the drainage tube via outlet 1004 into the peritoneal cavity for absorption by the body.

For example, as shown in FIG. 10C, the periphery of elastic membrane 1030 may be coupled to and sandwiched between flat portion 1021 of local constriction 1012/lower portion 1010 and an interior surface of upper portion 1006 of housing shell 1001, such that a middle portion of elastic membrane 1030 engages ridge 1020 in an assembled configuration. Accordingly, elastic membrane 1030 is exposed to both upstream (e.g., within inlet chamber 1024) and downstream (e.g., within groove 1026) pressure fluctuations, such that when either pressure increases, e.g., above a predetermined threshold, elastic membrane 1030 deforms outward, e.g., away from ridge 1020, thereby reducing fluid resistance of device 1000. Conversely, when either pressure decreases, elastic membrane 1030 contracts, e.g., towards ridge 1020, thereby increasing fluid resistance of device 1000.

The upper edge of ridge 1020 may have a circular profile, thereby defining inlet chamber 1024. Preferably, groove 1026 defines a fluidic channel lower than flat portion 1021 and ridge 1020 extends to a height above flat portion 1021 of local constriction 1012, such that the middle portion of elastic membrane 1030 is disposed at a height above the periphery of elastic membrane 1030, thereby providing a pre-loaded tension to elastic membrane 1030. The pre-loaded tension of elastic membrane 1030 may be selected to achieve a desired opening pressure of elastic membrane 1030, as described in further detail below. Alternatively, elastic membrane 1030 may have a high elastic modulus, e.g., thickness/stiffness, sufficient to be operable without a pre-loaded tension, such that elastic membrane 1030 has an overall flat configuration when there is no pressure acting on the interior surface of elastic membrane 1030, as shown in FIG. 10C. For example, in this embodiment, ridge 1020 may have the same height as flat portion 1021.

Elastic membrane 1030 preferably is constructed of a flexible biocompatible material, e.g., silicone, that requires a predetermined level of force to deform, and accordingly, disengage ridge 1020, e.g., responsive to pressure fluctuations across inlet 1002 and outlet 1004. The height of ridge 1020 and/or the thickness of elastic membrane 1030 may be selected to achieve a target predetermined pressure threshold required to cause elastic membrane 1030 to disengage ridge 1020, e.g., the opening pressure of elastic membrane 1030. Moreover, elastic membrane 1030 is leak-proof which ensures that fluid such as, e.g., CSF, traveling through the fluidic channel of device 1000 does not pass through opening 1008 of upper portion 1006 of housing shell 1001, and similarly, fluid within the body external to housing shell 1001 does not enter the fluidic channel of device 1000 via opening 1008.

As shown in FIG. 10C, elastic membrane 1030 at least partially defines the fluidic channel of device 1000 extending within housing shell 1001 between inlet 1002 and outlet 1004, and the addition of local constriction 1012 within the fluidic channel reduces the area of the fluid channel and increases fluidic resistance of the flow of fluid through the fluidic channel. For example, the engagement between ridge 1020 and the interior surface of elastic membrane 1030 divides the fluidic channel into an upstream fluidic channel inlet portion (including inlet chamber 1024) in fluid communication with inlet 1002, and a downstream fluidic channel outlet portion (including groove 1026) in fluid communication with outlet 1004, such that when ridge 1020 is engaged with the interior surface of elastic membrane 1030, e.g., at low CSF levels, fluid flow may not be permitted between the upstream and downstream fluidic channel inlet portions. Fluid flow will take place when the average pressure between inlet 1002 and outlet 1004 varies, e.g., exceeds a predetermined threshold, thereby causing elastic membrane 1030 to deform sufficiently to disengage ridge 1020 and permit fluid flow from inlet chamber 1024 across ridge 1020 into groove 1026.

Accordingly, the balance of forces between the atmospheric pressure forces applied against the exterior surface of elastic membrane 1030, e.g., through openings 1008, and pressure forces applied against the interior surface of elastic membrane 1030 as a result of the flow of CSF from the brain ventricles through inlet 1002 and into inlet chamber 1024, define the geometry of elastic membrane 1030, and accordingly, the size of the gap formed between the interior surface of elastic membrane 1030 and the upper edge of ridge 1020 upon deformation of elastic membrane 1030, and therefore the hydraulic resistance of the fluidic channel of device 1000. If upstream CSF pressure increases and/or if downstream pressure increases, e.g., due to progressive fibrosis formation at outlet 1004, the pressure acting the interior surface of elastic membrane 1030 will increase, thereby causing elastic membrane 1030 to deform even further and decreasing fluidic resistance to flow, which in turn will lower upstream pressures, restoring CSF levels to within acceptable physiological limits. Accordingly, the fluidic resistance of the fluidic channel of device 1000 is self-adjustable such that device 1000 will maintain a near-constant desired CSF (e.g., upstream pressure) at physiological levels at inlet end 1002 even if flow or pressure at outlet 1004 changes, e.g., without the need for magnetic parts to make adjustments in situ.

Advantageously, the self-adjustable hydrocephalus valve design of device 1000 may comprise an automatic anti-siphoning feature. For example, as described above, the downstream pressure can fluctuate significantly with changes in posture (e.g., standing up from a supine position can cause a drop in downstream pressure of up to 80 cm H₂O or more), thereby creating a vacuum effect on elastic membrane 1030 that may result in undesirable siphoning. In accordance with one aspect, the ratio of areas of elastic membrane 1030 that are exposed to upstream and downstream pressures may be selected to mitigate the undesirable siphoning effect. For example, the area of elastic membrane 1030 exposed to downstream pressure (e.g., within groove 1026) may be reduced, e.g., by increasing the width of the upper edge of ridge 1020 and/or flat portion 1021, while the area exposed to upstream pressure (e.g., within inlet chamber 1024) may be increased to thereby minimize the influence of downstream pressure on membrane deformation and valve resistance, effectively reducing siphoning effects when downstream pressure drops significantly.

While various illustrative embodiments of the invention are described above, it will be apparent to one skilled in the art that various changes and modifications may be made therein without departing from the invention. The appended claims are intended to cover all such changes and modifications that fall within the true scope of the invention.

What is claimed:

1. A device for the treatment of excess fluid pressure within a patient, the device comprising:
an inlet configured to be implanted to be in fluid communication with a first bodily area;
an outlet configured to be implanted in fluid communication with a second bodily area; and
an elastic membrane disposed between the inlet and the outlet and configured to be implanted at an implantation site within the patient, the elastic membrane comprising an interior surface that defines a fluidic channel that permits a flow of fluid from the inlet to the outlet, the elastic membrane further comprising an outer surface configured to be in direct contact with fluid or tissue surrounding the device at the implantation site, the elastic membrane configured to deform to change a volume of the fluidic channel when an average pressure between the inlet and the outlet varies, thereby varying fluidic resistance of the flow of fluid through the fluidic channel.

2. The device of claim 1, further comprising a housing defining the inlet and the outlet, the housing coupled to the elastic membrane to define the fluidic channel within the housing.

3. The device of claim 2, wherein the elastic membrane is disposed within a cavity of the housing, the cavity comprising one or more openings configured to expose the outer surface of the elastic membrane within the cavity to fluid or tissue surrounding the device at the implantation site.

4. The device of claim 2, wherein the first bodily area comprises an anterior chamber of an eye of the patient, wherein the second bodily area comprises a space in a distal portion or an orbital fat space of the eye, and wherein the elastic membrane is configured to deform to change the volume of the fluidic channel when the average pressure between the inlet and the outlet varies, thereby varying fluidic resistance of the flow of intraocular fluid through the fluidic channel.

5. The device of claim 4, wherein the housing has a radius of curvature selected to accommodate a radius of curvature of the eye.

6. The device of claim 4, wherein the housing is configured to be implanted under a conjunctiva.

7. The device of claim 4, further comprising a nozzle having an outlet end coupled to the inlet, and an inlet end configured to pass through a wall of an eye to communicate with the anterior chamber of the eye.

8. The device of claim 4, further comprising a drainage tube having a proximal end coupled to the outlet, and a distal region configured to be disposed within the space in the distal portion or the orbital fat space of the eye.

9. The device of claim 8, wherein the distal region of the drainage tube comprises one or more drainage holes.

10. The device of claim 8, further comprising a diffuser plate having a groove configured to receive a portion of the distal region of the drainage tube, the diffuser plate configured to be disposed on a scleral surface of the eye.

11. The device of claim 2, wherein the first bodily area comprises a ventricle of a brain of the patient, and wherein the elastic membrane is configured to deform to change the volume of the fluidic channel when the average pressure between the inlet and the outlet varies, thereby varying fluidic resistance of the flow of cerebrospinal fluid through the fluidic channel.

12. The device of claim 11, further comprising a nozzle having an outlet end coupled to the inlet, and an inlet end configured to be disposed within the ventricle of the brain.

13. The device of claim 11, further comprising a drainage tube having a proximal end coupled to the outlet, a distal region configured to pass through a wall of a peritoneum to communicate with a peritoneal cavity of the patient, and a length selected such that the distal region of the drainage tube extends from the outlet to the peritoneal cavity.

14. The device of claim 13, wherein the distal region of the drainage tube comprises one or more drainage holes.

15. The device of claim 2, further comprising a local constriction disposed within at least a portion of the fluidic channel to reduce the area of the fluidic channel and increase fluidic resistance of the flow of fluid through the fluidic channel.

16. The device of claim 15, wherein at least a portion of the local constriction is configured to engage the interior surface of the elastic membrane to divide the fluidic channel into a fluidic channel inlet portion in fluid communication with the inlet and a fluidic channel outlet portion in fluid communication with the outlet, and
wherein the elastic membrane is configured to deform and disengage the local constriction to permit fluid flow between the fluidic channel inlet portion and the fluidic channel outlet portion when the average pressure between the inlet and the outlet varies.

17. The device of claim 16, wherein the portion of the local constriction configured to engage the interior surface of the elastic membrane comprises a circular ridge protruding from an outer surface of the local constriction towards the elastic membrane, the circular ridge defining an inlet chamber of the fluidic channel inlet portion, such that, when the average pressure between the inlet and the outlet varies, fluid within the inlet chamber causes the elastic membrane to deform and disengage the circular ridge to permit fluid flow between the fluidic channel inlet portion and the fluidic channel outlet portion.

18. The device of claim 17, wherein the ridge has a height greater than a periphery of the elastic membrane to provide a pre-loaded tension to the elastic membrane.

19. The device of claim 17, wherein the housing comprises a circular shape, and wherein an upper portion of the housing comprises an inverted circular opening configured to expose the outer surface of the elastic membrane to fluid and tissue surrounding the device at the implantation site.

20. The device of claim 19, wherein the housing, the elastic membrane, and the local constriction are concentric.

21. The device of claim 17, wherein a first portion of the fluidic channel inlet portion is defined by a first inner surface of the local constriction, the first portion of the fluidic channel inlet portion fluidically coupling the inlet and the inlet chamber, and
wherein a first portion of the fluidic channel outlet portion is defined by the outer surface of the local constriction and the interior surface of the elastic membrane, the first portion of the fluidic channel outlet portion fluidically coupled to the outlet via a second portion of the fluidic channel outlet portion defined by a second inner surface of the local constriction.

22. The device of claim 21, wherein a ratio of an area of the first portion of the fluidic channel outlet portion and an area of the inlet chamber is selected to create an anti-siphoning effect.

23. The device of claim 21, wherein the first portion of the fluidic channel inlet portion is fluidically coupled to the inlet chamber via an opening at a lower portion of the inlet chamber.

24. The device of claim 21, wherein the outer surface of the local constriction defining the first portion of the fluidic channel outlet portion comprises a groove extending circumferentially around the circular ridge.

25. The device of claim 24, wherein the first portion of the fluidic channel outlet portion is fluidically coupled to the second portion of the fluidic channel outlet portion via an opening within a lower portion of the groove.

26. The device of claim 16, wherein the housing comprises a tubular shape extending from the inlet to the outlet, the housing comprising one or more openings configured to expose the outer surface of the elastic membrane to fluid and tissue surrounding the device at the implantation site.

27. The device of claim 26, wherein a longitudinal length of the one or more openings of the housing is larger than a longitudinal length of the local constriction.

28. The device of claim 26, wherein the one or more openings comprise a first opening at a first portion of the housing, and a second opening at a second portion of the housing opposite the first portion of the housing,
wherein the elastic membrane comprises a first elastic membrane and a second elastic membrane, the first elastic membrane exposed via the first opening and configured to engage with an upper portion of the local constriction, the second elastic membrane exposed via the second opening and configured to engage with a lower portion of the local constriction, and
wherein, when the average pressure between the inlet and the outlet varies, the first and second elastic membranes deform and disengage the upper and lower portions of the local constriction, respectively, to permit fluid flow between the fluidic channel inlet portion and the fluidic channel outlet portion.

29. The device of claim 16, wherein the housing comprises a rectangular shape extending from the inlet to the outlet, an upper portion of the housing comprising a proximal tapered portion, a distal tapered portion, and an opening between the proximal and distal tapered portions, the opening configured to expose the outer surface of the elastic membrane to fluid and tissue surrounding the device at the implantation site.

30. The device of claim 29, wherein a longitudinal length of the opening of the housing is larger than a longitudinal length of the local constriction.
